(19) United States
(12) Patent Application Publication
(10) Pub. No.: US 2025/0382573 A1
(43) Pub. Date: Dec. 18, 2025

(54) SERUM-FREE FORMULA FOR T CELL EXPANSION

(71) Applicant: Taiwan RedEye Biomedical Inc., Hisnchu City (TW)

(72) Inventors: Chao-Ling YAO, Hisnchu City (TW); Tsung-Yu Tseng, Hisnchu City (TW); Ya-Ling Chiang, Hisnchu City (TW); Shuo-Ting Yan, Hisnchu City (TW)

(73) Assignee: Taiwan RedEye Biomedical Inc., Hsinchu City (TW)

(21) Appl. No.: 18/745,684

(22) Filed: Jun. 17, 2024

(51) Int. Cl. C12N 5/0783

(52) U.S. Cl. CPC C12N 5/0636; C12N 2500/90; C12N 2501/2302; C12N 2501/2304; C12N 2501/2307; C12N 2501/231; C12N 2501/2315; C12N 2501/33

(57) ABSTRACT

Provided is a serum-free formula for T cell expansion, which comprises a basal serum-free medium and a cytokine combination, and the cytokine combination comprises IL-2, IL-4, IL-7, IL-10 and IL-15; wherein, based on a total volume of the basal serum-free medium, a content of IL-2 is from 5 ng/ml to 50 ng/ml, a content of IL-4 is from 5 ng/ml to 200 ng/ml, a content of IL-7 is from 5 ng/ml to 90 ng/ml, a content of IL-10 is from 5 ng/ml to 50 ng/ml and a content of IL-15 is from 5 ng/ml to 200 ng/ml. The serum-free formula for T cell expansion can exert the effects of expanding T cells, especially selectively expanding the CD3⁺CD8⁺ T cells, under a serum-free circumstance, thereby avoiding the uncertainty and the risks derived from the animal serum and promoting the development of adoptive cell therapy or immunotherapy.

### BACKGROUND OF THE INVENTION

### 1. Field of the Invention

The present invention relates to a formula for T cell expansion, particularly to a serum-free formula for T cell expansion.

### 2. Description of the Prior Arts

Cancer involves abnormal and uncontrollable cell proliferation with the potential to invade, even spread to other parts of the human body, so cancer is quite tricky to cure. Common cancer treatments include surgery, chemotherapy or radiation therapy. These treatments inevitably damage lots of normal cells during the process of eliminating cancer cells by physically removing the cancerous tissue, or using cytotoxic drugs or radiation which may damage all cells including cancer cells and normal cells. In addition, even though the patient is treated and the cancer cannot be detected for a period, recurrence of cancer is still possible, and it is usually a more difficult problem to deal with.

For pursuing cancer treatment with more efficacy and less damage to the human body, cell therapy has been developed and introduced for cancer treatment, which is called immunotherapy. Adoptive cell therapy (ACT) is an immunotherapy that can specifically recognize and kill cancer cells mainly by modified autologous T cells. The naïve T cells comprise the “naïve CD4⁺ T cells” (helper T cells) and the “naïve CD8⁺ T cells” (cytotoxic T cells, or killer T cells), and both the naïve CD4⁺ T cells and the naïve CD8⁺ T cells have CD3 protein on cell surface to form a complex for recognizing their targets. In the present invention, the naïve T cells are preliminarily selected by anti-CD3 antibody. Herein, the expressions of the “CD3⁺CD4⁺ T cells” and the “CD3⁺CD8⁺ T cells” are used to indicate activated naïve T cells, and the cells for recognizing and killing cancer cells in the adoptive cell therapy are mainly on the CD3⁺CD8⁺ T cells.

However, when conducting the adoptive cell therapy, the insufficient quantity of T cells for administrating to the patient is a critical problem, which limits the application of the adoptive cell therapy. For example, about 1×10⁹ of T cells are typically needed for administrating to the patient in the adoptive cell therapy, while there are only about 1×10⁶ of T cells per 1 milliliter (ml) in the human peripheral blood, and even 60 ml of blood only gives about 6×10⁷ of T cells, which still needs at least about 16 to 17 times of expansion to meet the quantity of T cells needed for the adoptive cell therapy. Therefore, the problem of how to effectively expand T cells becomes a key to unleash the potential of the adoptive cell therapy.

So far, a common way to expand T cells is to culture T cells in a suitable basal serum-free medium additionally supplemented with animal serum and interleukin-2 (IL-2). The animal serum, such as fetal bovine serum (FBS), is commonly added into culture medium for providing cells with several nutrients beneficial to cell growth. Nevertheless, the animal serum usually has the following drawbacks or risks: (1) the actual components of the animal serum are numerous, complicated and unknown; (2) different production batches of the animal serum may differ widely and lack reasonable reproducibility; (3) the purification steps of the animal serum are complicated and the quality tests for different production batches of the animal serum are time-consuming; and (4) the animal serum is usually heterogeneous and possibly carries pathogens, bacteria or viruses, which may cause infection or inflammation. In addition, in adoptive cell therapy using autologous T cells for human patients, the use of a xenogeneic material such as animal serum is disadvantageous.

Accordingly, there is still a need to research and develop a way that can expand T cells without using animal serum, so as to avoid the uncertainty and the risks derived from the animal serum, thereby boosting the progress of adoptive cell therapy or immunotherapy.

### SUMMARY OF THE INVENTION

In view of the problems in the prior art, one object of the present invention is to provide a way that can expand T cells under a serum-free circumstance.

Another object of the present invention is to provide a way that can selectively expand the CD3⁺CD8⁺ T cells, which indicates that a ratio of the proportion of the CD3⁺CD8⁺ T cells to the proportion of the CD3⁺CD4⁺ T cells is increased.

Yet another object of the present invention is to provide a way that can expand T cells, especially the CD3⁺CD8⁺ T cells, under a serum-free circumstance, in which comparable quantity of expanded T cells are obtained when compared to a conventional way of expanding T cells by using animal serum.

To achieve the foresaid objects, the present invention provides a serum-free formula for T cell expansion, which comprises a basal serum-free medium and a cytokine combination, and the cytokine combination comprises IL-2, interleukin-4 (IL-4), interleukin-7 (IL-7), interleukin-10 (IL-10) and interleukin-15 (IL-15); wherein, based on a total volume of the basal serum-free medium, a content of IL-2 is from 5 ng/ml to 50 ng/ml, a content of IL-4 is from 5 ng/ml to 200 ng/ml, a content of IL-7 is from 5 ng/ml to 90 ng/ml, a content of IL-10 is from 5 ng/ml to 50 ng/ml and a content of IL-15 is from 5 ng/ml to 200 ng/ml.

By adopting the cytokine combination with the specific kinds and contents of cytokines, the serum-free formula for T cell expansion of the present invention can not only expand T cells without using animal serum, but also selectively expand the CD3⁺CD8⁺ T cells, such that in the expanded T cells population, the proportion of the CD3⁺CD8⁺ T cells is obviously increased, which makes a ratio of the proportion of the CD3⁺CD8⁺ T cells to the proportion of the CD3⁺CD4⁺ T cells increase accordingly. Hence, the serum-free formula for T cell expansion of the present invention gives a way to expand T cells, especially the CD3⁺CD8⁺ T cells, without involving animal serum, thereby avoiding the uncertainty and the risks derived from the animal serum and promoting the development of adoptive cell therapy or immunotherapy.

In some embodiments of the present invention, the serum-free formula for T cell expansion further comprises insulin, and based on a total volume of the basal serum-free medium, a content of insulin is from 1 μg/ml to 10 μg/ml. By further adding the specific content of insulin, the serum-free formula for T cell expansion further has an effect of expanding the quantity of T cells to a level close to that using animal serum.

In some embodiments of the present invention, based on the total volume of the basal serum-free medium, the content of insulin is 1 from μg/ml to 9 μg/ml. In some embodiments of the present invention, based on the total volume of the basal serum-free medium, the content of insulin is from 1 g/ml to 8 μg/ml. In some embodiments of the present invention, based on the total volume of the basal serum-free medium, the content of insulin is from 1 g/ml to 7 μg/ml. In some embodiments of the present invention, based on the total volume of the basal serum-free medium, the content of insulin is from 1 g/ml to 6 μg/ml. In some embodiments of the present invention, based on the total volume of the basal serum-free medium, the content of insulin is from 2 μg/ml to 6 μg/ml.

In some embodiments of the present invention, based on the total volume of the basal serum-free medium, the content of IL-2 is from 10 ng/ml to 50 ng/ml. In some embodiments of the present invention, based on the total volume of the basal serum-free medium, the content of IL-2 is from 15 ng/ml to 50 ng/ml. In some embodiments of the present invention, based on the total volume of the basal serum-free medium, the content of IL-2 is from 20 ng/ml to 50 ng/ml. In some embodiments of the present invention, based on the total volume of the basal serum-free medium, the content of IL-2 is from 20 ng/ml to 45 ng/ml. In some embodiments of the present invention, based on the total volume of the basal serum-free medium, the content of IL-2 is from 20 ng/ml to 40 ng/ml.

In some embodiments of the present invention, based on the total volume of the basal serum-free medium, the content of IL-4 is from 10 ng/ml to 150 ng/ml. In some embodiments of the present invention, based on the total volume of the basal serum-free medium, the content of IL-4 is from 20 ng/ml to 120 ng/ml. In some embodiments of the present invention, based on the total volume of the basal serum-free medium, the content of IL-4 is from 30 ng/ml to 120 ng/ml. In some embodiments of the present invention, based on the total volume of the basal serum-free medium, the content of IL-4 is from 30 ng/ml to 100 ng/ml. In some embodiments of the present invention, based on the total volume of the basal serum-free medium, the content of IL-4 is from 35 ng/ml to 100 ng/ml. In some embodiments of the present invention, based on the total volume of the basal serum-free medium, the content of IL-4 is from 40 ng/ml to 100 ng/ml. In some embodiments of the present invention, based on the total volume of the basal serum-free medium, the content of IL-4 is from 50 ng/ml to 80 ng/ml.

In some embodiments of the present invention, based on the total volume of the basal serum-free medium, the content of IL-7 is from 10 ng/ml to 70 ng/ml. In some embodiments of the present invention, based on the total volume of the basal serum-free medium, the content of IL-7 is from 20 ng/ml to 70 ng/ml. In some embodiments of the present invention, based on the total volume of the basal serum-free medium, the content of IL-7 is from 20 ng/ml to 65 ng/ml. In some embodiments of the present invention, based on the total volume of the basal serum-free medium, the content of IL-7 is from 25 ng/ml to 60 ng/ml. In some embodiments of the present invention, based on the total volume of the basal serum-free medium, the content of IL-7 is from 30 ng/ml to 50 ng/ml.

In some embodiments of the present invention, based on the total volume of the basal serum-free medium, the content of IL-10 is from 10 ng/ml to 40 ng/ml. In some embodiments of the present invention, based on the total volume of the basal serum-free medium, the content of IL-10 is from 15 ng/ml to 40 ng/ml. In some embodiments of the present invention, based on the total volume of the basal serum-free medium, the content of IL-10 is from 15 ng/ml to 35 ng/ml.

In some embodiments of the present invention, based on the total volume of the basal serum-free medium, the content of IL-15 is from 10 ng/ml to 150 ng/ml. In some embodiments of the present invention, based on the total volume of the basal serum-free medium, the content of IL-15 is from 10 ng/ml to 125 ng/ml. In some embodiments of the present invention, based on the total volume of the basal serum-free medium, the content of IL-15 is from 10 ng/ml to 100 ng/ml. In some embodiments of the present invention, based on the total volume of the basal serum-free medium, the content of IL-15 is from 20 ng/ml to 100 ng/ml. In some embodiments of the present invention, based on the total volume of the basal serum-free medium, the content of IL-15 is from 30 ng/ml to 100 ng/ml. In some embodiments of the present invention, based on the total volume of the basal serum-free medium, the content of IL-15 is from 40 ng/ml to 100 ng/ml. In some embodiments of the present invention, based on the total volume of the basal serum-free medium, the content of IL-15 is from 50 ng/ml to 90 ng/ml. In some embodiments of the present invention, based on the total volume of the basal serum-free medium, the content of IL-15 is from 60 ng/ml to 80 ng/ml.

In some embodiments of the present invention, the basal serum-free medium is a serum-free medium for culturing leukocytes. One person skilled in the art knows that the leukocytes are part of the body's immune system, which help the body fight infection or diseases, and types of the leukocytes may be granulocytes (such as neutrophils, eosinophils and basophils), monocytes and/or lymphocytes (such as T cells and B cells).

In some embodiments of the present invention, the leukocytes comprise nature killer cells (NK cells), dendritic cells (DCs), macrophages, T cells and/or B cells.

In some embodiments of the present invention, the basal serum-free medium comprises L-glutamine, human albumin and human transferrin.

In some embodiments of the present invention, the basal serum-free medium may be X-VIVO™ 15 medium or AIM-V™ medium. In some embodiments of the present invention, the basal serum-free medium may be X-VIVO™ 15 medium.

In some embodiments of the present invention, the serum-free formula for T cell expansion further comprises a T cell activator, which activates naïve T cells. Naïve T cells comprise the naïve CD4⁺ T cells and the naïve CD8⁺ T cells. One person skilled in the art knows that the activation of naïve T cells indicates transforming the naïve CD4⁺ T cells and the naïve CD8⁺ T cells to the activated CD4⁺ T cells (marked as CD3⁺CD4⁺ T cells) and the activated CD8⁺ T cells (marked as CD3⁺CD8⁺ T cells), respectively.

In some embodiments of the present invention, the T cell activator comprises peptides that activate CD3 protein and peptides that activate CD28 protein. In some embodiments of the present invention, the T cell activator comprises anti-CD3 antibody and anti-CD28 antibody. In some embodiments of the present invention, the T cell activator may be magnetic microbeads coated with anti-CD3 antibody and anti-CD28 antibody. In some embodiments of the present invention, the T cell activator may be CD3/CD28 Dynabeads.

In this specification, a range represented by “a lower-limit value to an upper-limit value” indicates that the range is more than or equal to the lower-endpoint value and less than or equal to the upper-endpoint value, unless otherwise specified. For example, “from 5 ng/ml to 50 ng/ml” indicates that a range of “more than or equal to 5 ng/ml and less than or equal to 50 ng/ml”.

Other objectives, advantages and novel features of the present invention will become more apparent from the following detailed description.

### DETAILED DESCRIPTION OF THE PREFERRED EMBODIMENTS

Hereinafter, some embodiments and test examples are exemplified to illustrate the implementation and the effects of the present invention. One person skilled in the art can easily realize the advantages and effects of the present invention in accordance with the contents of the specification. Various modifications and variations could be made in order to practice or apply the present invention without departing from the spirit and scope of the invention.

### Example 1: Serum-Free Formula for T Cell Expansion

A commercial medium named X-VIVO™ 15 medium (hereinafter referred to as X-VIVO 15), purchased from Lonza; item number: 04-418Q, was used as a basal serum-free medium, and then suitable amounts of the cytokines of IL-2 (purchased from PeproTech; item number: 200-02), IL-4 (purchased from PeproTech; item number: 200-04), IL-7 (purchased from PeproTech; item number: 200-07), IL-10 (purchased from PeproTech; item number: 200-10) and IL-15 (purchased from PeproTech; item number: 200-15) were added into the basal serum-free medium, so as to obtain the serum-free formula for T cell expansion of Example 1 (hereinafter referred to as E1 medium). Based on a total volume of the basal serum-free medium, a content of IL-2 was 30.8 ng/ml, a content of IL-4 was 67.3 ng/ml, a content of IL-7 was 40.3 ng/ml, a content of IL-10 was 26.8 ng/ml and a content of IL-15 was 70 ng/ml.

### Example 2: Serum-Free Formula for T Cell Expansion (with Insulin)

The preparation of Example 2 was similar to Example 1. Specifically, the X-VIVO 15 was also used as a basal serum-free medium, and then suitable amounts of the cytokines of IL-2, IL-4, IL-7, IL-10 and IL-15, and suitable amount of insulin (purchased from Novo Nordisk; item number: A10AC01) were added into the basal serum-free medium, so as to obtain the serum-free formula for T cell expansion of Example 2 (hereinafter referred to as E2 medium). Based on a total volume of the basal serum-free medium, a content of IL-2 was 30.8 ng/ml, a content of IL-4 was 67.3 ng/ml, a content of IL-7 was 40.3 ng/ml, a content of IL-10 was 26.8 ng/ml, a content of IL-15 was 70 ng/ml, and a content of insulin was 4.39 μg/ml.

### Comparative Example 1: Basal Serum-Free Medium

The basal serum-free medium used in Examples 1 and 2, i.e., the X-VIVO 15, were set as Comparative Example 1 in the following Test Examples (hereinafter referred to as CE1 medium).

### Comparative Example 2: Formula for T Cell Expansion Containing Animal Serum

The preparation of Comparative Example 2 was similar to Example 1. Specifically, the X-VIVO 15 was also used as a basal serum-free medium, and then suitable amounts of the cytokines of IL-2, IL-4, IL-7, IL-10 and IL-15, and suitable amount of FBS (purchased from Cytiva; item number: SH30071.03) were added into the basal serum-free medium, so as to obtain the Formula for T cell expansion of Comparative Example 2 (hereinafter referred to as CE2 medium). Based on a total volume of the basal serum-free medium, a content of IL-2 was 30.8 ng/ml, a content of IL-4 was 67.3 ng/ml, a content of IL-7 was 40.3 ng/ml, a content of IL-10 was 26.8 ng/ml, a content of IL-15 was 70 ng/ml, and a content of FBS was 10 vol % (volume percent).

### Preparation Example: Selection of the CD3⁺ T Cells

50 ml of the human cord blood was collected and then centrifuged at a condition of 2000 revolutions per minute (rpm) for 20 minutes to separate and give a plasma layer, a buffy coat cells layer and a red blood cells (RBC) layer. Afterward, the buffy coat cells layer was collected and added into Ficoll buffer (purchased from Cytiva; item number: 17144003) under volume ratio of 1:1, and then density centrifugation was conducted at a condition of 2000 rpm for 40 minutes to obtain a supernatant layer, a mononuclear cells layer, a Ficoll buffer layer and an RBC layer. Next, the mononuclear cells layer was collected and centrifugated under a condition of 2000 rpm for 10 minutes, and then the supernatant was collected and mixed with CD3 microbeads (purchased from Miltenyi Biotec; item number: 130-097-043), i.e., magnetic microbeads coated with anti-CD3 antibody, to obtain a mixture. Next, VarioMACS separator (purchased from Miltenyi Biotec; item number: 130-090-282) was used to screen out the CD3 T cells bound with the CD3 microbeads in the mixture through magnetic force, and then the CD3 T cells were obtained for the following Test Examples.

### Test Example: Evaluation of the Effect of T Cell Expansion Under Serum-Free Circumstance

The CD3⁺ T cells obtained in the Preparation Example were cultured in 24-well plates respectively with the E1 medium, the E2 medium, the CE1 medium and the CE2 medium under a condition of 5% CO₂ at 37° C., and the original cell density of the CD3 T cells was 1×10⁵ cell/ml/well, and the medium volume of each well was 1 ml. Meanwhile, CD3/CD28 Dynabeads (purchased from Gibco™; item number: 11161D) were also added into the foresaid mediums as a T cell activator, which transformed the CD3⁺ T cells to the CD3⁺CD4⁺ T cells and the CD3⁺CD8⁺ T cells during expansion of T cells, and a content of the CD3/CD28 Dynabeads was 2 μl/ml/1×10⁵ cells.

After 3.5 days of culture, the cells in each group, i.e., cultured in the E1 medium, the E2 medium, the CE1 medium and the CE2 medium, were supplemented with 1 ml of the corresponding fresh medium and passaged for continuous expansion. After another 3.5 days of culture (7 days of culture in total), the cells in each group were collected for the following Analyses (1) to (3), which could demonstrate the expansion results for T cells among different groups. It was understood that the cells after above-mentioned 7 days of culture contained the following types of T cells at the same time: the CD3 T cells, the CD3⁺CD4⁺ T cells and the CD3⁺CD8⁺ T cells. In the following Analyses (1) to (3), the group adopting the E1 medium for T cell expansion was marked as E1 hereinafter; the group adopting the E2 medium for T cell expansion was marked as E2 hereinafter; the group adopting the CE1 medium for T cell expansion was marked as CE1 hereinafter; and the group adopting the CE2 medium for T cell expansion was marked as CE2 hereinafter.

Analysis (1): a Ratio of the Proportion of the CD3⁺CD8⁺ T cells to the proportion of the CD3⁺CD4⁺ T Cells

After the foresaid 7 days of culture, the cell number of each group was counted by a hemocytometer, and the cells with cell density of 1×10⁵ cell/tube of E1, E2, CE1 and CE2 were collected and centrifuged under a condition of 2000 rpm for 10 minutes. Next, after the excess medium was removed, the cells were mixed with an anti-CD3 antibody with FITC (purchased from BD Pharmingen™; item number: 561807), an anti-CD4 antibody with PerCP (purchased from BD Pharmingen™; item number: 566924), and an anti-CD8 antibody with PE (purchased from BD Pharmingen™; item number: 555367), respectively for 30 minutes reaction. After the reaction, the solutions were centrifuged at 2000 rpm for 10 minutes for removing the excess antibodies, to obtain samples ready for analysis. The samples of E1, E2, CE1 and CE2 were analyzed by a flow cytometer (purchased from BD Bioscience; item number: 23-13347-00) to obtain the cell number of different types of T cells, which could be used to obtain the ratio of the proportion of the CD3⁺CD8⁺ T cells to the proportion of the CD3⁺CD4⁺ T cells (hereinafter referred to as “CD8/CD4”). The results of averaged CD8/CD4 of E1, E2, CE1 and CE2 were listed in the following Table 1 as the results listed in Table 1 were based on four repeated experiments (n=4).

TABLE 1
The results of averaged CD8/CD4 of E1, E2, CE1 and CE2.

| Groups | Averaged CD8/CD4 |
|---|---|
| E1 | 0.76 ± 0.14 |
| E2 | 0.73 ± 0.08 |
| CE1 | 0.53 ± 0.11 |
| CE2 | 0.55 ± 0.06 |

According to the results in Table 1, the results of averaged CD8/CD4 of E1 and E2 were about 0.76 and 0.73, respectively, while both the results of averaged CD8/CD4 of CE1 and CE2 were about 0.55 or less. When compared to CE1 (only using basal serum-free medium for T cell culture and expansion) and CE2 (using medium containing animal serum for T cell culture and expansion), the proportions of the CD3⁺CD8⁺ T cells in E1 and E2 were obviously higher than the proportions of the CD3⁺CD4⁺ T cells. Therefore, it was verified that the serum-free formula for T cell expansion of the present invention could selectively expand the CD3⁺CD8⁺ T cells, thereby benefiting the development of adoptive cell therapy or immunotherapy as the CD3⁺CD8⁺ T cells played a main role in these therapies.

Analysis (2): The Proportion of the CD3⁺CD8⁺ T Cells

The preparations of the samples of E1, E2, CE1 and CE2 were the same to Analysis (1). Afterward, the samples of E1, E2, CE1 and CE2 were also analyzed by the flow cytometry to obtain the cell number of different types of T cells, which could be used to obtain the proportion of the CD3⁺CD8⁺ T cells in the total CD3⁺ T cells. The results of E1, E2, CE1 and CE2 were listed 2 in the following Table 2. Also, the relative cell number of the CD3⁺CD8⁺ T cells were calculated and listed in the following Table 2 by normalization in which the cell number of the CD3⁺CD8⁺ T cells of E1 was defined as 100%. The results listed in Table 2 were based on four repeated experiments (n=4).

TABLE 2
The results of the proportion and the relative cell number
of the CD3⁺CD8⁺ T cells of E1, E2, CE1 and CE2.

| | | Relative cell |
|---|---|---|
| | Proportion of the | number of the |
| Groups | CD3⁺CD8⁺ T cells (%) | CD3⁺CD8⁺ T cells (%) |
| E1 | 50.4 ± 9.1 | 100 ± 0 |
| E2 | 47.6 ± 9.4 | 131 ± 59 |
| CE1 | 39.1 ± 8.9 | 36 ± 24 |
| CE2 | 37.3 ± 9.7 | 156 ± 145 |

According to the results in Table 2, the proportion of the CD3⁺CD8⁺ T cells of E1 and E2 was about 50% in the total CD3⁺ T cells, while the proportion of the CD3⁺CD8⁺ T cells of CE1 and CE2 was only about 40% in the total CD3⁺ T cells, which was consistent with the effect of selectively expanding the CD3⁺CD8⁺ T cells of E1 and E2 shown in Analysis (1).

Besides, with reference to the results of E1, E2 and CE1, the relative numbers of the CD3⁺CD8⁺ T cells of E1 and E2 were 100% and about 131%, respectively, which gave at least about 3 times higher than that of CE1 (about 36%). Hence, it was verified that the serum-free formula for T cell expansion of the present invention not only could expand the CD3⁺CD8⁺ T cells under a serum-free circumstance, but also obtain much higher quantity of the CD3⁺CD8⁺ T cells compared to a case only using basal serum-free medium for T cell expansion.

Besides, with reference to the results of E2 (using a medium comprising insulin) and CE2 (using a medium comprising FBS), the relative number of the CD3⁺CD8⁺ T cells of E2 was about 131% and the relative number of the CD3⁺CD8⁺ T cells of CE2 was about 156%, which showed comparable results between E2 and CE2. Hence, it was verified that the serum-free formula for T cell expansion of the present invention could obtain comparable quantity of the CD3⁺CD8⁺ T cells when compared to a conventional way by using animal serum, but the uncertainty and risks derived from animal serum were eliminated.

Analysis (3): The Expansion Fold of the CD3⁺CD8⁺ T Cells

The preparations of the samples of E2, CE1 and CE2 were the same to Analysis (1). Afterward, the samples of E2, CE1 and CE2 were also analyzed by the flow cytometry to obtain the cell number of different types of T cells, and the proportion of the CD3⁺CD8⁺ T cells in the total CD3⁺ T cells were specifically focused. Besides, before the foresaid 7 days of culture, the initial proportion of the CD3⁺CD8⁺ T cells in the total CD3⁺ T cells of E2, CE1 and CE2 were also analyzed by the flow cytometry. Then, a ratio of the initial proportion of the CD3⁺CD8⁺ T cells (before the foresaid 7 days of culture) to the final proportion of the CD3⁺CD8⁺ T cells (after the foresaid 7 days of culture) could be obtained and represented the expansion fold of the CD3⁺CD8⁺ T cells. The results of E2, CE1 and CE2 were listed in the following Table 3. The results listed in Table 3 were based on four repeated experiments (n=4).

TABLE 3
The results of the expansion fold of the
CD3⁺CD8⁺ T cells of E2, CE1 and CE2.

| Groups | Expansion fold of the CD3⁺CD8⁺ T cells |
|---|---|
| E2 | 5.6 ± 2.3 |
| CE1 | 1.0 ± 1.1 |
| CE2 | 5.7 ± 2.3 |

According to the results in Table 3, the expansion fold of the CD3⁺CD8⁺ T cells of E2 was about 5.6, which was obviously higher than that of CE1 (about 1.0). This demonstrated that the effect of expanding the CD3⁺CD8⁺ T cells of E2 was much better than CE1 and was consistent with the results shown in Analysis (2).

Besides, with reference to the results of E2 and CE2, the expansion fold of the CD3⁺CD8⁺ T cells of E2 (about 5.6) and CE2 (about 5.7) were almost the same, which indicated that E2 (using a medium comprising insulin) could obtain comparable quantity of the CD3⁺CD8⁺ T cells when compared to CE2 (using a medium comprising FBS). This was consistent with the results shown in Analysis (2).

Accordingly, based on the foresaid Analyses (1) to (3), it is verified that even under a serum-free circumstance, the serum-free formula for T cell expansion of the present invention can not only expand the CD3⁺CD8⁺ T cells, but also selectively expand the CD3⁺CD8⁺ T cells, so the proportion of the CD3⁺CD8⁺ T cells is obviously increased in the expanded T cells population.

In summary, as the present invention adopts the specific kinds and contents of cytokines, the serum-free formula for T cell expansion of the present invention gives a way to expand T cells, especially the CD3⁺CD8⁺ T cells, without involving animal serum, thereby avoiding the uncertainty and the risks derived from the animal serum and promoting the development of adoptive cell therapy or immunotherapy.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A serum-free formula for T cell expansion, comprising a basal serum-free medium and a cytokine combination, and the cytokine combination comprising interleukin-2, interleukin-4, interleukin-7, interleukin-10 and interleukin-15; wherein, based on a total volume of the basal serum-free medium, a content of interleukin-2 is from 5 ng/ml to 50 ng/ml, a content of interleukin-4 is from 5 ng/ml to 200 ng/ml, a content of interleukin-7 is from 5 ng/ml to 90 ng/ml, a content of interleukin-10 is from 5 ng/ml to 50 ng/ml and a content of interleukin-15 is from 5 ng/ml to 200 ng/ml.

2. The serum-free formula for T cell expansion as claimed in claim 1, wherein the serum-free formula for T cell expansion further comprises insulin, and based on the total volume of the basal serum-free medium, a content of insulin is from 1 μg/ml to 10 μg/ml.

3. The serum-free formula for T cell expansion as claimed in claim 1, wherein based on the total volume of the basal serum-free medium, the content of interleukin-2 is from 10 ng/ml to 50 ng/ml.

4. The serum-free formula for T cell expansion as claimed in claim 2, wherein based on the total volume of the basal serum-free medium, the content of interleukin-2 is from 10 ng/ml to 50 ng/ml.

5. The serum-free formula for T cell expansion as claimed in claim 1, wherein based on the total volume of the basal serum-free medium, the content of interleukin-4 is from 10 ng/ml to 150 ng/ml.

6. The serum-free formula for T cell expansion as claimed in claim 2, wherein based on the total volume of the basal serum-free medium, the content of interleukin-4 is from 10 ng/ml to 150 ng/ml.

7. The serum-free formula for T cell expansion as claimed in claim 1, wherein based on the total volume of the basal serum-free medium, the content of interleukin-7 is from 10 ng/ml to 70 ng/ml.

8. The serum-free formula for T cell expansion as claimed in claim 2, wherein based on the total volume of the basal serum-free medium, the content of interleukin-7 is from 10 ng/ml to 70 ng/ml.

9. The serum-free formula for T cell expansion as claimed in claim 1, wherein based on the total volume of the basal serum-free medium, the content of interleukin-10 is from 10 ng/ml to 40 ng/ml.

10. The serum-free formula for T cell expansion as claimed in claim 2, wherein based on the total volume of the basal serum-free medium, the content of interleukin-10 is from 10 ng/ml to 40 ng/ml.

11. The serum-free formula for T cell expansion as claimed in claim 1, wherein based on the total volume of the basal serum-free medium, the content of interleukin-15 is from 10 ng/ml to 150 ng/ml.

12. The serum-free formula for T cell expansion as claimed in claim 2, wherein based on the total volume of the basal serum-free medium, the content of interleukin-15 is from 10 ng/ml to 150 ng/ml.

13. The serum-free formula for T cell expansion as claimed in claim 1, wherein the basal serum-free medium is a serum-free medium for culturing leukocytes.

14. The serum-free formula for T cell expansion as claimed in claim 13, wherein the leukocytes comprise a nature killer cell, a dendritic cell, a macrophage, a T cell or a B cell.

15. The serum-free formula for T cell expansion as claimed in claim 1, wherein the basal serum-free medium comprises L-glutamine, human albumin and human transferrin.

16. The serum-free formula for T cell expansion as claimed in claim 1, wherein the serum-free formula for T cell expansion further comprises a T cell activator, which activates naïve T cells.

17. The serum-free formula for T cell expansion as claimed in claim 16, wherein the T cell activator comprises peptides that activate CD3 protein and peptides that activate CD28 protein.

18. The serum-free formula for T cell expansion as claimed in claim 16, wherein the T cell activator is CD3/CD28 Dynabeads.
